(19) United States
(12) Patent Application Publication
(10) Pub. No.: US 2006/0229555 A1
(43) Pub. Date: Oct. 12, 2006

(54) Safety syringe with a needle seat

(76) Inventor: Wei-Shui Wu, Taichung Hsien (TW)

Correspondence Address: HDSL, 4331 STEVENS BATTLE LANE, FAIRFAX, VA, 22033, US

(21) Appl. No.: 11/102,783

(22) Filed: Apr. 11, 2005

(51) Int. Cl. A61M 5/00

(52) U.S. Cl. 604/110; 128/919

(57) ABSTRACT

A safety syringe with a needle seat includes a syringe, a rod, a needle seat and a needle sheath. The needle seat is installed inside the container part of the syringe in resist status. After the medicine is injected, the holding part of the push rod will enter the base of the needle seat to make the wall of the base become a conical wall gradually, and then make the holding part and concave-buckle part of the rod connect with the linking part and positioning part of the base. After connection, the outer diameter size of the base is still a little smaller than the inner diameter size of the circular-convex part. Therefore, after use and pull out the rod, the needle seat will be departed from the container part and stored inside the syringe to avoid people being stung by the discard syringe.

### BACKGROUND OF THE INVENTION

1. Field of the Invention

The present invention relates to a safety syringe with a needle seat, and more particularly to a syringe and a needle seat can be stored inside the syringe of a safety syringe.

2. Description of Related Art

A safe medical needle apparatus is helpful for patient, medical personnel, environment and public safety. It is not only involved in dealing with the medical needle apparatus but also can reduce the unsafe injection behavior such as reusing the medical needle apparatus, reusing the needle, or incorrect operation. In order to collect the medical waste, some medical and health facilities discard the medical waste into an anti-seepage, anti-puncture package or an airtight container.

After use a syringe, the needle can be put into a safe place if a safe medical needle apparatus is used. On the other hand, the safe medical needle apparatus can avoid the needle-stick injuries for medical personnel while they putting a needle into a needle sheath.

The inventor of the present invention takes the drawbacks of the above mention safe medical needle apparatus as a lesson and improves its efficient, thereby achieving this safety syringe.

### SUMMARY OF THE INVENTION

The invention is to provide a safety syringe with a needle seat, which can prevent the pollution of the environment and reduce the volume of medical waste.

Another, this invention is to provide a safety syringe with a needle seat, wherein the needle sheath can be stuck in the syringe after use.

Further, this invention is to provide a safety syringe with a needle seat, wherein the needle seat can be put inside the container part of the syringe after use.

Still further, this invention is to provide a safety syringe with a needle seat, wherein many connection points connect the push rod and the neck; the push rod and the neck can be broken apart after use.

Accordingly, this invention is to provide a safety syringe with a needle seat comprising a syringe, a push rod, a needle seat set and a needle sheath.

The syringe has a tube room for storing medicine. The top of the tube room is formed a wedge and the bottom of the tube room is set a circular-convex part inside. The circular-convex part and bottom are formed a container part. The inner container part is shrunk into a cone along the bottom from the circular-convex part, and a perforation is set along the outer edge of the syringe in the container part.

The push rod has a top being set a holding part, a concave-buckle part, a block part and a neck. The push rod is set a collar, and the back end of the block part that is below the collar will be wedged in the wedge part and can not be dropped out anymore after the push rod push the tube room of the syringe forward.

The needle seat set in the container part of the syringe includes a base with changing wall, an close-knit part and a sheath part. The shape of the upper plane of the base is like having a long and short axis, and is like an oval shape. And the total length of the outer surround of the upper plane is smaller than the total length of the inner surround of the circular-convex part. The shape of the lower plane of the base is a circle. The base is downward concave to set an interlinking part, and the top of the interlinking part is set a plurality of positioning parts, and the there is a hole penetrating through the needle seat set on the needle seat. The hole can firm up the needle and make the needle to be interlinked with the hole in the base.

The needle sheath with hollow-cone shape is to provide an sheath for the needle. The outer circumference of the needle sheath is set a plurality of bulges.

The needle seat uses the base to penetrate through the cube space of the syringe, and uses the two end of the long axis of the base upper plane to prop the lower edge of the circular-convex part, thereby forming a container part inside the syringe in resist status. Moreover, the close-knit part of the needle seat knit the perforation of the syringe together with the lower plane of the base, and the collar of the push rod can be knit together with the tube room of the syringe.

### BRIEF DESCRIPTION OF THE DRAWINGS

Other objects and aspects of the present invention will become apparent from the following description of embodiments with reference to the accompanying drawing in which:

FIG. 1 is an exploded view of the first embodiment of the safety syringe with a needle seat of the present invention;

FIGS. 2 (A) to (D) are enlarged schematic views of different embodiments of the needle seat;

FIG. 3 is an exploded view of the second embodiment of the safety syringe with a needle seat of the present invention;

FIG. 4 is the schematic view of the third embodiment of the safety syringe with a needle seat of the present invention;

FIGS. 5 (A) to (C) are motion schematic views of the fourth embodiment of the safety syringe with a needle seat of the present invention;

FIG. 6 (A) is a motion schematic view showing that the holding part of the push rod is going to enter the needle seat;

FIG. 6 (B) is a schematic view showing that the holding part of the push rod has entered the needle seat;

FIG. 6 (C) is a schematic view showing that the push rod and the needle seat are going to depart from the container part of syringe;

FIG. 6 (D) is a schematic view showing that the push rod and the needle seat have depart completely from the container part of syringe; and

FIG. 7 is a schematic view showing that the push rod of the safe syringe has been pulled out from the syringe, and the push rod is broken apart from the neck.

### DESCRIPTION OF THE PREFERRED EMBODIMENT

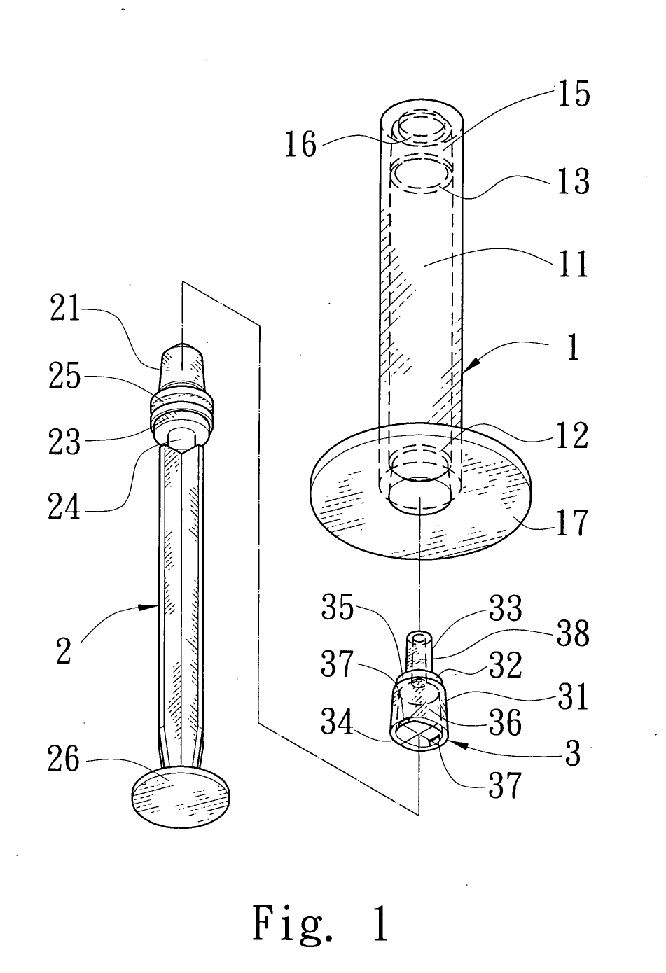

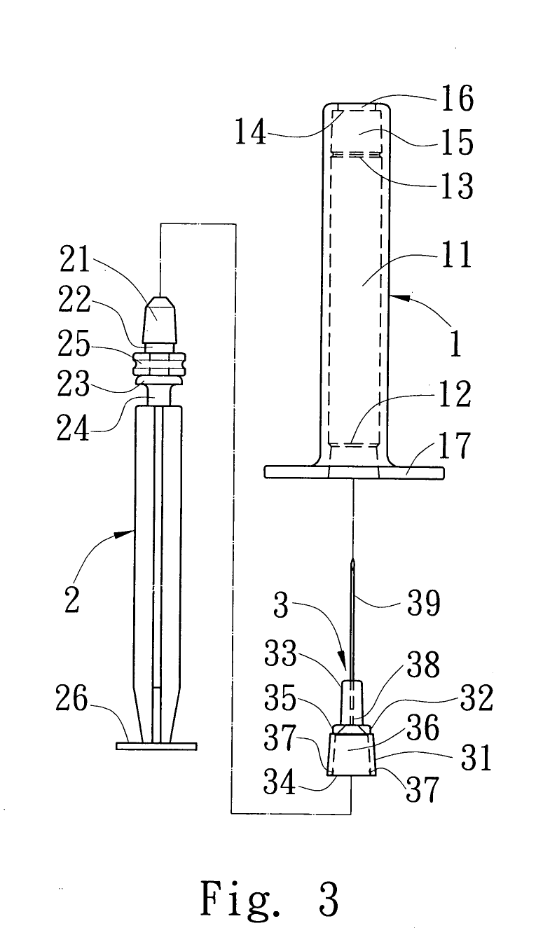

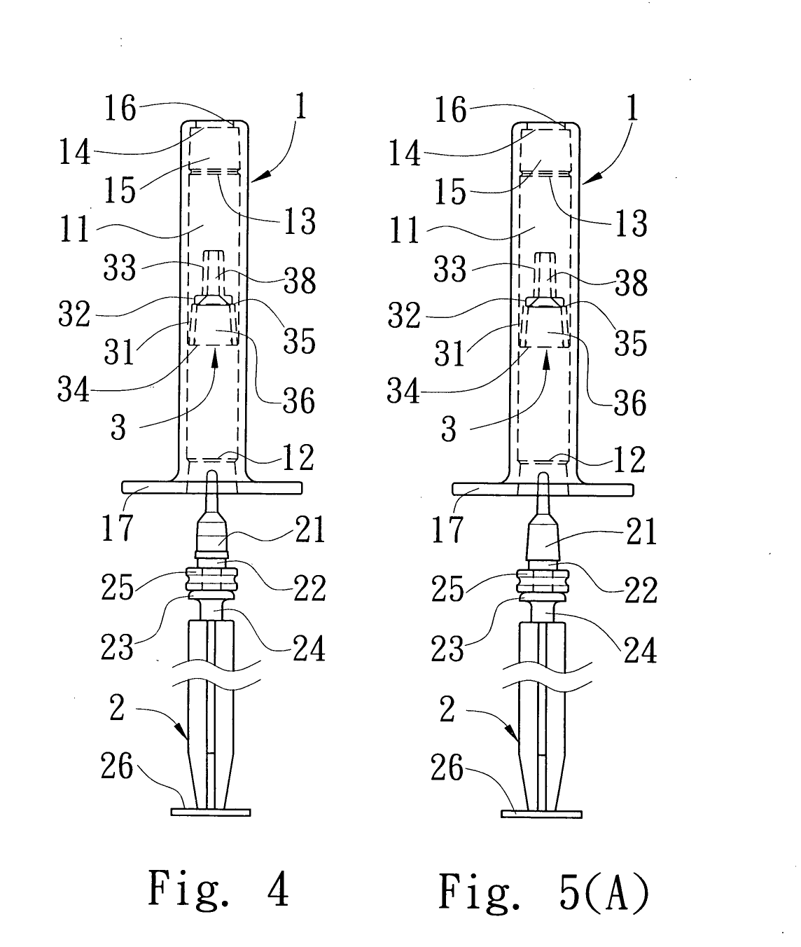

By referencing FIG. 1, FIG. 3 and FIG. 4, this invention provide a safety syringe with a needle seat includes a syringe 1, a push rod 2, a needle seat 3 and a needle sheath 4.

The syringe 1 has a tube room 11 for storing medicine. the top of the tube room 11 is formed a wedge 12 and the bottom 14 of the tube room 11 is set a circular-convex part 13 inside. The circular-convex part 13 and the bottom 14 are formed a container part 15 which is shrunk into a cone along the bottom 14 from the circular-convex part 13, and a perforation 16 is set along the outer edge of the syringe 1 in the container part 15.

The push rod 2 has the top being set a holding part 21, a concave-buckle part 22 (only shown in FIG. 3), a block part 23, a neck 24 and a collar 25. The back end of the block part 23 that is below the collar 25 will be wedged in the wedge 12 and can not be dropped out anymore after the push rod 2 pushes the tube room 11 of the syringe 1 forward. The FIG. 3 is the second embodiment of the push rod 2 of this invention, and FIG. 4 is the third embodiment of the push rod 2 of this invention;

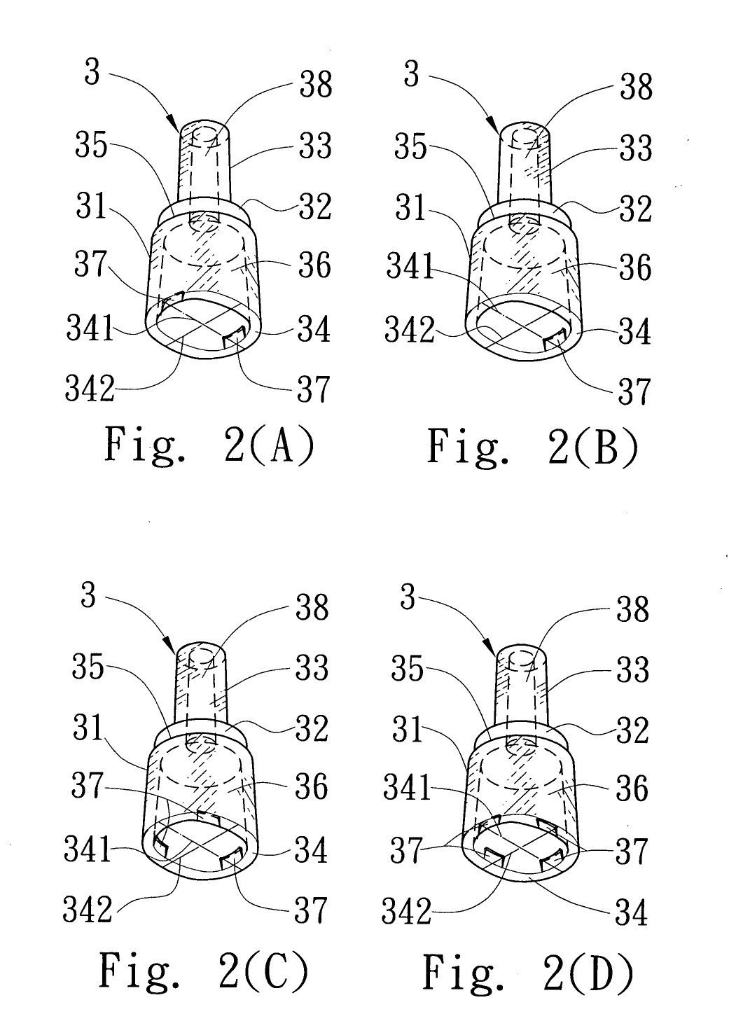

The needle seat 3 is set in the container part 15 of the syringe 1, which includes a base 31 with changing wall, a close-knit part 32 and a sheath part 33. The shape of the upper plane 34 of the base 31 is like having a long axis 341 and short axis 342, and is like an oval shape. The total length of the outer surround of the upper plane 34 is smaller than the total length of the inner surround of the circular-convex 13 part. The shape of the lower plane 35 of the base 31 is a circle. The base 31 is downward concave to set an interlinking part 36, and the top of the interlinking part 36 is set a plurality of positioning parts 37. There is a hole 38, which penetrate through the needle seat 3 set on the needle seat 3. The hole 38 can firm up the needle 39 and make the needle 39 to be interlinked with the hole 38 in the base 31 as shown in FIG. 2 (A) to FIG. 2 (D).

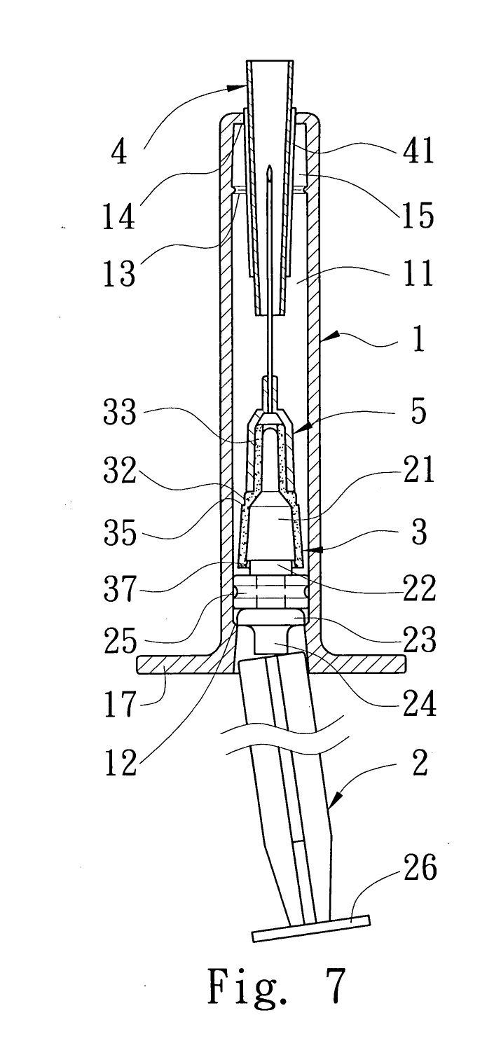

The needle sheath 4 with hollow-cone shape is to provide an sheath for the needle 39. The outer circumference of the needle sheath 4 is set a plurality of bulges 41 as shown in FIG. 7. The needle sheath 4 can be the shape that the top and bottom 14 are interlinked with each other or the shape that only has one open section. The present embodiment uses the case that the top and bottom 14 are interlinked with each other as an illustration.

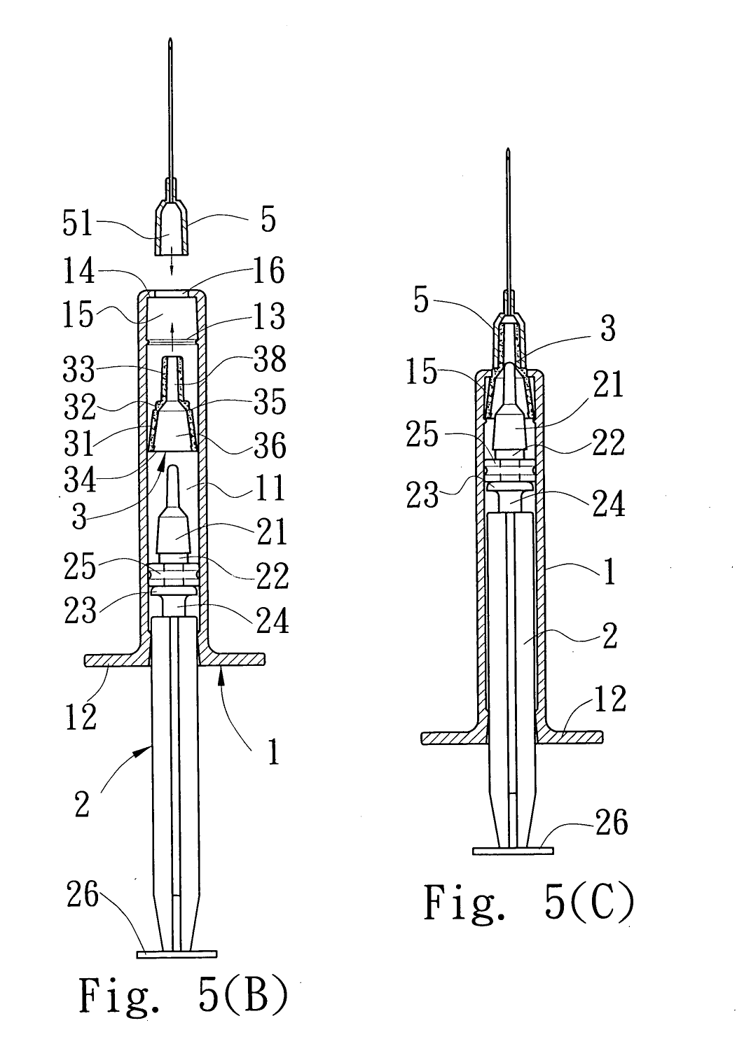

By referencing FIGS. 5 (A) to (C), the needle seat 3 uses the base 31 to penetrate through the cube space 11 of the syringe 1, and uses the two end of the long axis 341 of the upper plane 34 of the base 31 to prop the lower edge of the circular-convex part 13, thereby forming a container part 15 inside the syringe 1 in resist status. Moreover, the close-knit part 32 of the needle seat 3 knit the perforation 16 of the syringe 1 together with the lower plane of the base 31, and the collar 25 of the push rod 2 can be knit together with the tube room 11 of the syringe 1. The sheath part 33 of the needle seat 3 is provided for the needle bed 5, and the needle bed 5 uses the normal standard one. However, user can use another standard of needle 39 according to requirement, and the needle bed 5 is linked with the sheath part 33 of the needle seat 3 by the join part 51.

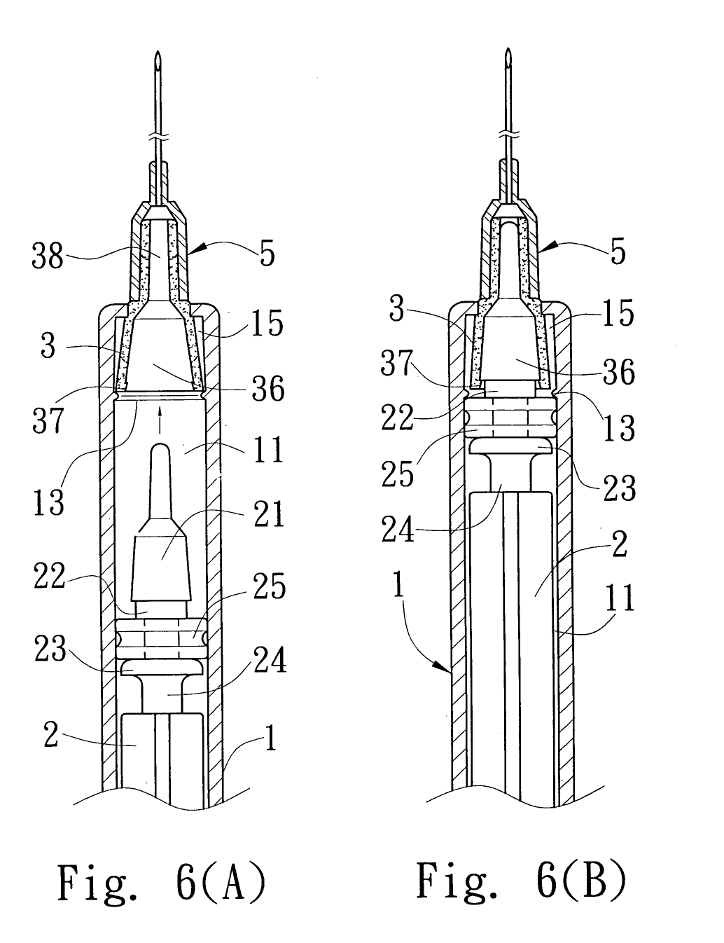

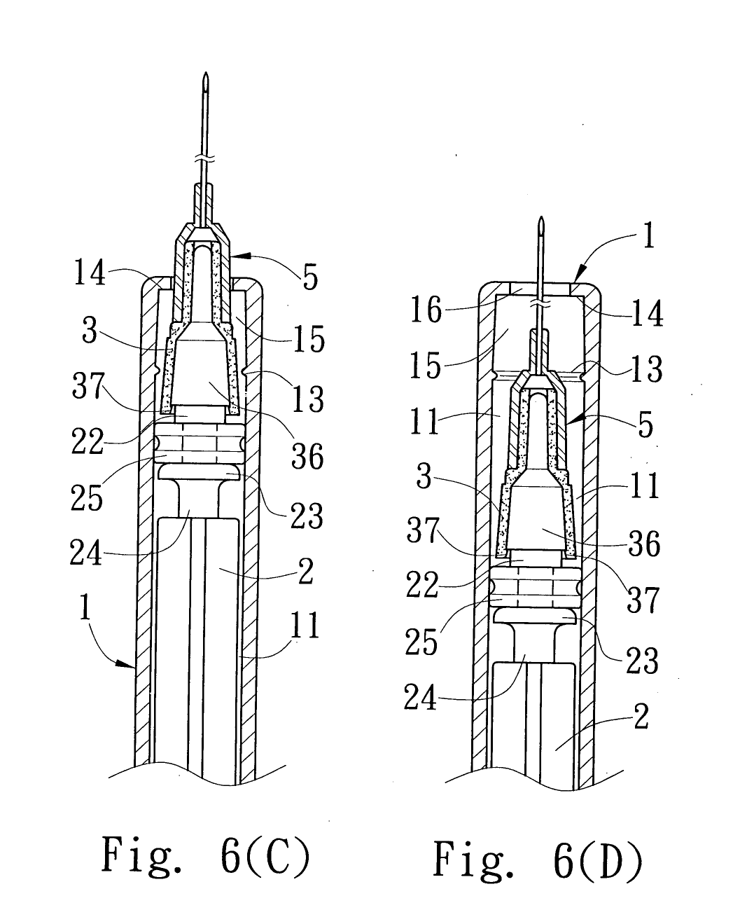

In order to describe this invention further, the schematic views of embodiment of this invention are shown in FIG. 6 (A) to FIG. 6 (D). While the safety syringe with a needle seat being used, the collar 25 can be knit with the tube room 11 of the syringe 1 closely because the collar 25 of the push rod 2 is made by rubber or silica gel. User can use first and middle finger to prop the prop part 17 of the syringe 1, and slightly press the press part 26 of the push rod 2 by thumb, and then the medicine inside the syringe will be injected outside from the needle 39. After medicine being injected completely, the push force will make the holding part 21 of the push rod 2 enter the interlinking part 36 of the base 31.

While the holding part 21 of the push rod 2 is entering the base 31, the changing wall of the base 31 will become a conical wall gradually to make that the changing wall between the upper plane 34 and the lower plane 35 of the base 31 knit closely with the holding part 21, and thereby the holding part 21 of the push rod 2 will be interlinked with the interlinking part 36 of the base 31 by pressing and friction forces. After the holding part 21 of the push rod 2 enters the interlinking part 36 of the base 31, the positioning part 37 of the needle seat 3 will be embedded in the concave-buckle part 22 of the push rod 2. At the same time, after the holding part 21 of the push rod 2 enters the base 31, the outer diameter size of the upper plane 34 is a little smaller than the inner diameter size of the circular-convex part 13. Therefore, after user pulls out the push rod 2, the needle seat 3 is departed from the container part 15 and stored inside the tube room 11 of the syringe 1 as shown in FIG. 7.

FIG. 2 (A) is an enlarged schematic view of the needle seat of this invention, and FIG. 2 (B) to FIG. 2 (D) is the schematic view of other embodiments of the needle seat. The positioning part 37 is set on the top of the interlinking part 36. One or a plurality of the positioning parts 37 can be set according to the requirement to strengthen the linking force that is used for the needle seat 3 to depart from the container part 15 of the syringe 1.

In the syringe 1 of this invention, the inner diameter of the container 15 is larger than the inner diameter of the perforation 16, thereby forming a step. Therefore, the base 31 can be fit into the container part 15 and the perforation 16 completely when the base 31 is inside the syringe 1. The knit-close design between the container part 15 and the perforation 16 can avoid the medicine being leaking while injecting. Moreover, the wedge 12 located near the prop part 17 of the syringe 1 is formed into a step shape with the top of the syringe 1. Therefore, after the safety syringe with a needle seat is used, if user pushes the push rod 2 to the wedge 12, the block part 23 of the push rod 2 will be locked at the wedge 12 to make the push rod can not be away from the syringe 1. Furthermore, some interlinking points interlink the push rod 2 and the neck 24, and they can be broken apart after used as shown in FIG. 7.

Summing up the above, the present invention not only innovates space, but also improves much above-mention efficiency to the prior art.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A safety syringe with a needle seat, comprising:
a syringe having a tube room for storing medicine,
wherein the top of the tube room is formed a wedge and the bottom of the tube room is set a circular-convex part inside,
wherein the circular-convex part and the bottom are formed a container part;
a push rod, whose top is set a holding part, a concave-buckle part, a block part and a neck;
a needle seat set in the container part of the syringe, which includes a base with a changing wall, an close-knit part and a sheath part;
wherein the shape of the lower plane of the base is a circle, the base is downward concave to set an interlinking part, and the top of the interlinking part is set a plurality of positioning parts, and there is a hole penetrating through the needle seat set on the needle seat; and
a needle sheath with hollow-cone shape to provide an sheath for the needle,
whereby, as the holding part of the push rod is entering the base, the changing wall of the base will become a conical wall gradually to make that the changing wall between the upper plane and the lower plane of the base knit closely with the holding part, and thereby the holding part of the push rod will be interlinked with the interlinking part of the base by pressing and friction forces, and after the holding part of the push rod enter the interlinking part of the base, the positioning part of the needle seat will be embedded in the concave-buckle part of the push rod, and, as the holding part of the push rod enters the base, the outer diameter size of the upper plane of the base is a little smaller than the inner diameter size of the circular-convex part, and then after user pulls out the push rod, the needle seat is departed from the container part and stored inside the tube room of the syringe.

2. The safety syringe according to claim 1, wherein the hole of the needle seat can be joined a needle.

3. The safety syringe according to claim 1, wherein the sheath part of the needle seat can be set a needle bed.

4. The safety syringe according to claim 1, wherein the base of the needle seat is downward concave to set the interlinking part, and the top of the interlinking part is set one or a plurality of the positioning parts.

5. The safety syringe according to claim 1, wherein the inner container part is shrunk into a cone along the bottom from the circular-convex part, and a perforation is set along the outer edge of the syringe in the container part.

6. The safety syringe according to claim 1, wherein the push rod is set a collar, and the back end of the block part that is below the collar will be wedged in the wedge part and can not be dropped out anymore after the push rod push the tube room of the syringe forward.

7. The safety syringe according to claim 1, wherein the shape of the upper plane of the base is like having a long and short axis, and is like an oval shape.

8. The safety syringe according to claim 1, wherein the total length of the outer surround of the upper plane of the base is smaller than the total length of the inner surround of the circular-convex part.

9. The safety syringe according to claim 1, wherein the hole can firm up the needle and make the needle to be interlinked with the hole in the base.

10. The safety syringe according to claim 1, wherein the outer circumference of the needle sheath is set a plurality of bulges.

11. The safety syringe according to claim 1, wherein the needle seat uses the base to penetrate through a cube space of the syringe, and uses the two end of the long axis of the upper plane of the base to prop the lower edge of the circular-convex part, thereby forming the container part inside the syringe in resist status.

12. The safety syringe according to claim 6, wherein the close-knit part of the needle seat knit the perforation of the syringe together with the lower plane of the base, and the collar of the push rod can be knit together with the tube room of the syringe to provide user can slightly press the press part of the push rod by thumb, and then the medicine inside the syringe will be injected outside from the needle, and after medicine being injected completely, the push force will make the holding part of the push rod enter the interlinking part of the base,
